(19) United States
(12) Patent Application Publication
(10) Pub. No.: US 2013/0028979 A1
(43) Pub. Date: Jan. 31, 2013

(54) Stretch Marks Cream

(71) Applicant: Carmen Maria Lechin, Houston, TX (US)

(72) Inventor: Carmen Maria Lechin, Houston, TX (US)

(21) Appl. No.: 13/562,846

(22) Filed: Jul. 31, 2012

Related U.S. Application Data
(60) Provisional application No. 61/513,657, filed on Jul. 31, 2011.

(51) Int. Cl. A61K 35/64; A61P 17/02

(52) U.S. Cl. 424/526

(57) ABSTRACT

A topical composition and method for making, including the steps of placing cow's tallow in water and heating; allowing the heating tallow to float to the top of the water; filtering the floating portion of the tallow from the rest of the water; placing the floating portion of tallow obtained from the filtering into a container containing the bee's wax, camphor, and alum powder; maintaining the mixture at low heat; allowing all four ingredients to have blended; cooling the mixture from the low heat; and refrigerating the mixture. The temperature of heating the cow's tallow is sufficient to render the four ingredients blendable.

### BACKGROUND OF THE INVENTION

1. Field of the Invention

The present invention relates to topical compositions for application on the skin, in a particular example, a composition or cream, which when applied to the skin of an individual, improves or erases the appearance of stretch marks and scars.

2. Description of the Prior Art

Current creams used to treat stretch marks are based on compounds containing mainly collagen and elastin. These are complex, long protein molecules that are too big to be absorbed by the skin; therefore, they cannot repair and fill the gaps in the matrix of collagen and elastin that supports the dermis and epidermis.

### SUMMARY

In one example, disclosed is a topical composition including: (a) cow's tallow, 2000 grams in proportion; (b) bee's wax, 800 grams in proportion; (c) camphor, 100 grams in proportion; and (d) alum in powder, 400 grams in proportion; and wherein the weight proportions of cow's tallow, bee's wax, camphor, and alum are prior to processing and mixing; and where the weight proportion of the cow's tallow is prior to a heating of the cow's tallow in water and a floating tallow extraction.

In a further example, the cow's tallow provides a mixture of 70% to 80% of stearin and palmitin, and corresponding 20% to 30% of oleic.

In a further example, the alum powder is hydrated potassium aluminum sulfate having the formula KAl(SO4)2.12H2O.

In a further example, the composition further includes a cream base.

In another example, disclosed is a method for treating skin comprising application of the topical composition.

In a further example, the method for treating skin includes heating the topical composition and applying to the desired skin area.

In a further example, the method for treating skin includes heating the topical composition and applying to a desired skin area nightly for about twelve (12) weeks or less.

In a further example, the method for treating skin includes application of the topical composition, allowing the composition to dry on the skin and removing the next day.

In another example, disclosed is a method for making a topical composition, including: placing cow's tallow in water and heating; allowing the heating tallow to float to the top of the water; filtering the floating portion of the tallow from the rest of the water; preparing a container containing bee's wax, camphor, and alum powder; placing the floating portion of tallow obtained from the filtering into the container containing the bee's wax, camphor, and alum powder, thereby forming a mixture; maintaining the mixture at low heat, below 149 degrees Fahrenheit but above room temperature; allowing all four ingredients to have blended; cooling the mixture from the low heat; and refrigerating the mixture; and where the temperature of heating the cow's tallow is sufficient to render the four ingredients blendable.

In a further example of the method, the temperature of heating the cow's tallow is about 149 degrees Fahrenheit.

In a further example of the method, (a) the cow's tallow is 2000 grams in proportion; (b) the bee's wax is 800 grams in proportion; (c) the camphor is 100 grams in proportion; and (d) the alum in powder is 400 grams in proportion; and where the weight proportion of the cow's tallow is prior to the heating of the cow's tallow in the water.

In a further example of the method, the cow's tallow provides a mixture of 70% to 80% of stearin and palmitin, and corresponding 20% to 30% of oleic.

In a further example of the method, the alum powder is hydrated potassium aluminum sulfate having the formula KAl(SO4)2.12H2O.

In a further example of the method, the mixing, heating, and cooling steps are adapted to produce exhibition of a candle-wax hardness in the resulting composition.

In a further example, the method includes adapting the mixture for delivery into a cream base.

In further examples, disclosed are topical compositions made according to the above methods.

### DETAILED DESCRIPTION OF EXAMPLES OF THE INVENTION

From my research, I have found or discovered that stretch marks form from the breakdown and stretching of collagen fiber and elastin, which lays beneath the top layer of the skin (epidermis). This creates gaps between the matrix of collagen and elastin that supports the dermis and epidermis. In some people, when the skin stretches, the fibers of collagen and elastin break down. This leaves gaps that, when seen through the epidermis, produce the effect of stretch marks. This is associated with the rupture of the vessels producing breakage of the capillaries, along with an inflammatory response, which causes the associated change in coloration seen in the acute phase of the stretch mark.

I decided to study each product separately and to learn how stretch marks develop. Now I have come to understand how they work in the regeneration of the skin, separately and in combination.

### 1. Composition

In an example, the topical composition of the present invention comprises a combination of: (a) cow's tallow, 2000 grams in proportion; (b) bee's wax, 800 grams in proportion; (c) camphor, 100 grams in proportion; and (d) alum in powder, 400 grams in proportion. The weight proportions of these ingredients (cow's tallow, bee's wax, camphor, alum) are prior to processing and mixing. The weight proportion of the cow's tallow is prior to boiling and tallow extraction. In this context, “boiling” refers to heating the tallow sufficiently in water for the tallow to float to the top of the water.

In a preferred example, the topical composition of the present invention comprises a combination of: (a) cow's tallow, 2000 grams; (b) bee's wax, 800 grams; (c) camphor, 100 grams; and (d) alum in powder, 400 grams.

In one example, the cow's tallow provides a mixture of 70% to 80% of stearin and palmitin, and corresponding 20% to 30% of oleic. The cow's tallow (stearin, palmitin, oleic) provides a means for lubricating the skin. These three ingredients provide a means for assisting in the production of collagen and elastin. Cow's tallow has properties as a skin lubricant. It helps in the healing of the skin, which is very important in the process of erasing stretch marks and scars.

Bee's Wax: is an Anti-inflammatory and helps the cicatrisation process in the skin. It also works as a hardening agent. One of the problems that Stretch Marks present on the skin is the lost of the flexibility or tightening. Also, once the skin is broken by the stretch marks, the skin experiences an inflammatory process. Bee's wax heals the skin, decreasing inflammation while tightening the skin. In one example, the bee's wax provides a means for hardening the topical composition. In one example, the bee's wax provides a means for reducing inflammation of the skin. In one example, the bee's wax provides a means for helping the cicatrisation process in the skin.

Camphor: is a vasodilator that improves the capillary blood flow circulation of the skin. Good blood flow circulation is vital for the absorption of the ingredients and the delivery of nutrients and oxygen, along with the formation of new capillaries. In one example, the camphor of the topical composition is a means for improving capillary blood flow circulation of the skin.

Powder of Alum: has antiseptic properties and is an astringent, but most importantly it also helps in the cicatrisation of the skin. Astringents help prepare the skin for the absorption of creams. The Powder of Alum also closes the gap created by the stretch mark by causing a retraction of the subcutaneous tissue. In one example, the powder of Alum provides a means (or, an additional means) for helping the cicatrisation process in the skin. In one example, the powder of Alum provides a means for reducing sepsis. In one example, the powder of Alum provides a means for contraction of body tissues. In one example, the powder of Alum provides a means for enabling absorption of creams by the skin.

In one example, the Powder of Alum is the hydrated potassium aluminum sulfate (Potassium alum) with the formula KAl(SO4)2.12H2O.

### 2. Method of Manufacture

In an example of the present invention, the product is prepared of small enough molecules so as to be absorbed by the skin. In one example, the proportions of the ingredients are established to perform one or more functions: increase the synthesis of collagen and elastin, increase blood circulation, decrease inflammation and produce a retraction of the broken collagen and elastin matrix to repair and reduce the stretch marks gap.

In an example of making the product of the present invention, the steps as follows are performed. One, extract the cow's tallow by placing the tallow in water and heating to 149 degrees Fahrenheit. The 149 degrees Fahrenheit was seen to be sufficient to prepare the tallow to render the four ingredients blendable. Therefore, in one example, the heating temperature of the cow's tallow in water is that which is sufficient to render the four ingredients blendable (extracted tallow, bee's wax, camphor, and alum powder). Once the tallow floats to the top of the water, then proceed to filter the floating portion of the tallow from the rest of the water. Prepare a container containing the bee's wax, camphor, and alum powder. Place the floating portion of tallow that was obtained from the filter into the container containing the bee's wax, camphor, and alum powder. Maintain the mixture in the container at low heat, below 149 degrees Fahrenheit but above room temperature. Wait until all four ingredients have blended. Proceed to allow the mixture to cool from the low heat, then refrigerate.

In one example, this method has worked when using (a) cow's tallow, 2000 grams; (b) bee's wax, 800 grams; (c) camphor, 100 grams; and (d) alum in powder, 400 grams and when placed in a suitably sized container for purposes of low heat mixing, cooling from the low heat, and refrigeration.

In one example, the mixing, heating, and cooling method is adapted to produce a resulting product that exhibits a hard consistence, like that of a candle.

In one example, when this product is prepared it acquires a hard consistence, like that of a candle. When the product is heated, it can be applied to the skin to exert its effect. After a few weeks, you can notice significant improvement of the stretch marks and/or disappearance of the marks. Scars improve significantly and sometimes can be erased.

### 3. Method of Application—Method of Use

In an example of using the product of the present invention, heat small portions of the product to liquefy. Apply the product to the desired skin area. In one example, apply nightly. Repeat application of the product nightly until the stretch marks or scars are erased.

In one example, the product of the present invention is heated and applied nightly to the affected area of the skin, for about twelve (12) weeks or less.

In one example, the product of the present invention is allowed to dry on the skin after application and removed the next day after application.

In an example, the product of the present invention is adapted to be delivered in a cream base.

In an example, the product of the present invention is adapted for treatment of stretch marks. In an example, the product of the present invention is adapted to be delivered in a cream base. In one example, the product of the present invention is heated and applied nightly to the affected area of the skin. The product is allowed to dry on the skin after application and removed the next day after application. In one example, the product of the present invention is heated and applied nightly to the affected area of the skin, for about twelve (12) weeks or less.

In an example, the product of the present invention is adapted for treatment of scars from burns. In an example, the product of the present invention is adapted for treatment of scars from third degree burns. In an example, the product of the present invention is adapted to be delivered in a cream base. In one example, the product of the present invention is heated and applied nightly to the affected area of the skin. The product is allowed to dry on the skin after application and removed the next day after application. Application is repeated daily (or, in one example, nightly) until results are achieved.

In an example, the product of the present invention is adapted for treatment of keloids. In an example, the product of the present invention is adapted to be delivered in a cream base. In one example, the product of the present invention is heated and applied nightly to the affected area of the skin. The product is allowed to dry on the skin after application and removed the next day after application. Application is repeated daily (or, in one example, nightly) until results are achieved.

In an example, the product of the present invention is adapted for facial use. In an example, the product of the present invention is adapted to be delivered in a cream base. In one example, the product of the present invention is applied daily (or, in one example, night and day) to the face. In one example, the product of the present invention is applied daily (or, in one example, night and day) to the face with your facial routine.

### 4. Testing

In testing, the combination of the four ingredients appears to improve or erase stretch marks and scars. Testing was conducted on my daughter, myself, and others.

Thus, the foregoing description is presented for purposes of illustration and description, and is not intended to limit the invention to the forms disclosed herein. Consequently, variations and modifications commensurate with the above teachings and the teaching of the relevant art are within the spirit of the invention. Such variations will readily suggest themselves to those skilled in the relevant structural or mechanical art. Further, the embodiments described are also intended to explain the best mode for practicing the invention, and to enable others skilled in the art to utilize the invention and such or other embodiments and with various modifications required by the particular applications or uses of the invention.

I claim:

1. A topical composition comprising: (a) cow's tallow, 2000 grams in proportion; (b) bee's wax, 800 grams in proportion; (c) camphor, 100 grams in proportion; and (d) alum in powder, 400 grams in proportion; and wherein the weight proportions of cow's tallow, bee's wax, camphor, and alum are prior to processing and mixing; and wherein the weight proportion of the cow's tallow is prior to a heating of the cow's tallow in water and a floating tallow extraction.

2. The topical composition of claim 1 wherein the cow's tallow provides a mixture of 70% to 80% of stearin and palmitin, and corresponding 20% to 30% of oleic.

3. The topical composition of claim 1 wherein the alum powder is hydrated potassium aluminum sulfate having the formula KAl(SO4)2.12H2O.

4. The topical composition of claim 1 further comprising a cream base.

5. A method for treating skin comprising application of the topical composition of claim 1.

6. A method for treating skin comprising heating the topical composition of claim 1 and applying to the desired skin area.

7. A method for treating skin comprising heating the topical composition of claim 1 and applying to the desired skin area nightly for about twelve (12) weeks or less.

8. A method for treating skin comprising application of the topical composition of claim 1, allowing the composition to dry on the skin and removing the next day.

9. A method for making a topical composition, comprising:
placing cow's tallow in water and heating;
allowing the heating tallow to float to the top of the water;
filtering the floating portion of the tallow from the rest of the water;
preparing a container containing bee's wax, camphor, and alum powder;
placing the floating portion of tallow obtained from the filtering into the container containing the bee's wax, camphor, and alum powder, thereby forming a mixture;
maintaining the mixture at low heat, below 149 degrees Fahrenheit but above room temperature;
allowing all four ingredients to have blended;
cooling the mixture from the low heat; and
refrigerating the mixture; and
wherein the temperature of heating the cow's tallow is sufficient to render the four ingredients blendable.

10. The method of claim 9 wherein the temperature of heating the cow's tallow is about 149 degrees Fahrenheit.

11. The method of claim 9 wherein: (a) the cow's tallow is 2000 grams in proportion; (b) the bee's wax is 800 grams in proportion; (c) the camphor is 100 grams in proportion; and (d) the alum in powder is 400 grams in proportion; and wherein the weight proportion of the cow's tallow is prior to the heating of the cow's tallow in the water.

12. The method of claim 9 wherein the cow's tallow provides a mixture of 70% to 80% of stearin and palmitin, and corresponding 20% to 30% of oleic.

13. The method of claim 9 wherein the alum powder is hydrated potassium aluminum sulfate having the formula KAl(SO4)2.12H2O.

14. The method of claim 9 wherein the mixing, heating, and cooling steps are adapted to produce exhibition of a candle-wax hardness in the resulting composition.

15. The method of claim 9 further comprising adapting the mixture for delivery into a cream base.

16. A topical composition made according to the method of claim 9.

17. A topical composition made according to the method of claim 10.

18. A topical composition made according to the method of claim 11.

19. A topical composition made according to the method of claim 12.

20. A topical composition made according to the method of claim 13.

21. A topical composition made according to the method of claim 14.

22. A topical composition made according to the method of claim 15.
